(19) United States
(12) Patent Application Publication
(10) Pub. No.: US 2003/0114388 A1
(43) Pub. Date: Jun. 19, 2003

(54) Auxiliary agents for ophthalmic operation

(76) Inventors: Tomiya Mano, Osaka (JP); Takatoshi Maeno, Hyogo (JP)

Correspondence Address: WENDEROTH, LIND & PONACK, L.L.P., 2033 K STREET N. W., SUITE 800, WASHINGTON, DC, 20006-1021, US

(21) Appl. No.: 10/182,441

(22) PCT Filed: Dec. 27, 2000
(86) PCT No.: PCT/JP00/09290

(30) Foreign Application Priority Data
Jan. 27, 2000 (JP) 2000-18682

(51) Int. Cl. A61K 38/04; A61K 31/4709

(52) U.S. Cl. 514/19; 514/314

(57) ABSTRACT

An auxiliary method for an ophthalmic operation and a method for prevention of easy clotting of intraoperative bleeding, in which (2R,4R)-4-methyl-1-[N²((RS)-3-methyl-1,2,3,4-tetrahydro-8-quinolinesulfonyl)-L-arginyl]-2-piperidinecarboxylic acid, a hydrate thereof, or pharmaceutically acceptable salts thereof is used. According to the present invention, treatment for intraoperative bleeding becomes easier, so that loads of a patient and an operator can be reduced and incidence of intraoperative iatrogenic retinal tear can be lowered.

### TECHNICAL FIELD

The invention relates to an auxiliary method for ophthalmic operation and a method for preventing easy clotting of intraoperative bleeding in which a particular class of arginine amide compound, a hydrate thereof, or a pharmaceutically acceptable salt thereof is used.

### BACKGROUND ART

For controlling intraoperative bleeding during vitreous surgery for a patient with proliferating diabetic retinopathy, means for preventing blood flow out of a ruptured part of a vessel have been used which include application of ultra high perfusion pressure or addition of thrombin to an irrigating solution. “Proliferation” means that neovascularization arises around the retina. Due to fragile tendency of the neogenic vessels, bleeding is easily generated, and if the bleeding is not treated, it may lead to diminution of vision and loss of eyesight. In addition, treatment of coagulation mass of flowed out blood is still a very difficult problem for an operator.

A blood coagulation mass adheres to the retina very firmly, and the adhesion becomes still firmer by the thrombin perfusion for prevention of intraoperative bleeding and the like. It is necessary to carry out detachment treatment of the blood coagulation mass adhered. However, even if the treatment is performed very carefully, intraoperative iatrogenic retinal tear may sometimes be caused during the treatment of coagulation mass adhered firmly to the retina. In addition, it is also pointed out that retinal disorder may be caused by the perfusion pressure. As for intraocular operations, unlike ordinary treatments under vascular surgical operations, if an iatrogenic disorder is caused by the surgery, the disorder may affect postoperative QOL(s) such as diminution of vision, and accordingly, operations in this field should be conducted delicately. For these reasons, intraoperative administrations of anticoagulants or thrombolytic agents have not normally been considered.

It is known that arginine amide compounds have highly specific preventive effect on thrombin in a mammalian body, and are useful as therapeutic and preventive treatment of thrombosis, or as platelet aggregation inhibitors. As for their application in the ophthalmologic filed, Japanese Patent Unexamined Publication (KOKAI) No. 6-9401 discloses a use thereof as inhibitors against fibrin formation in an anterior chamber. However, the publication neither suggests or describes an application as an auxiliary medicament for ophthalmic operation.

### DISCLOSURE OF THE INVENTION

The inventors of the present invention conducted various researches to provide a medicament which can be used for controlling intraoperative bleeding during an ophthalmic operation as mentioned above. As a result, they first found that a particular class of arginine amide compounds, one of anticoagulants that have been believed to be inapplicable for treatment of bleeding during ophthalmic operations, had excellent desired effect and achieved the present invention.

The gists of the present invention are an auxiliary method for an ophthalmic operation and a method for prevention of easy clotting of intraoperative bleeding, in which (2R,4R)-4-methyl-1-[N²((RS)-3-methyl-1,2,3,4-tetrahydro-8-quinolinesulfonyl)-L-arginyl]-2-piperidinecarboxylic acid, a hydrate thereof, or a pharmaceutically acceptable salt thereof is used.

According to preferred embodiments of the present invention, the compound is (2R,4R)-4-methyl-1-[N²((RS)-3-methyl-1,2,3,4-tetrahydro-8-quinolinesulfonyl)-L-arginyl]-2-piperidinecarboxylic acid monohydrate; the compound is an auxiliary medicament for the treatment of coagulation mass intraoperatively generated during an ophthalmic operation; the ophthalmic operation is for proliferating diabetic retinopathy; the ophthalmic operation is a vitreous surgery; and the compound is administered as an intraocular irrigating solution or an eye drop.

Further gists of the present invention include: a method for controlling bleeding during an ophthalmic operation by using (2R,4R)-4-methyl-1-[N²((RS)-3-methyl-1,2,3,4-tetrahydro-8-quinolinesulfonyl)-L-arginyl]-2-piperidinecarboxylic acid, a hydrate thereof, or pharmaceutically acceptable salts thereof; and a method for reducing an incidence of intraoperative iatrogenic retinal tear by using (2R,4R)-4-methyl-1-[N²((RS)-3-methyl-1,2,3,4-tetrahydro-8-quinolinesulfonyl)-L-arginyl]-2-piperidinecarboxylic acid, a hydrate thereof, or pharmaceutically acceptable salts thereof.

### BEST MODE FOR CARRYING OUT THE INVENTION

The present invention will be explained in detail. The compound used as the active ingredient of the medicament of the present invention is represented by the following formula:

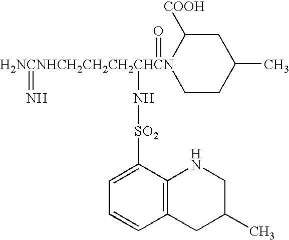

and is a known compound as described in Japanese Patent Publication (KOKOKU) Nos. (Sho) 61-48829 and (Hei) 1-35000 and the like. The compound can be readily prepared by the methods described in Japanese Patent Publication (KOKOKU) Nos. (Sho) 61-48829 and (Hei) 1-35000 and the like or similar methods thereto. The aforementioned patent publications suggest that the compound of the above formula has highly specific inhibitory action against thrombin in a mammalian body, and is useful for treatment or prevention of thrombosis or useful as a platelet aggregation inhibitor. A use in the ophthalmic field of said compound as an inhibitor of fibrin formation in an anterior chamber is described in Japanese Patent Unexamined Publication (KOKAI) No. (Hei) 6-9401. However, the publications neither suggests nor teaches that the compound is useful as an auxiliary medicament for ophthalmic operation according to the present invention.

As an active ingredient of the auxiliary medicament for ophthalmic operation of the present invention, the compound in free form may be used. Any hydrate or solvate thereof, or pharmaceutically acceptable salts of the aforementioned compound or any hydrate or solvate thereof may also be used.

According to the present invention, a monohydrate of the aforementioned compound may be most preferably used. In particular, an preferable compound includes (2R,4R)-4-methyl-1-[N²((RS)-3-methyl-1,2,3,4-tetrahydro-8-quinolinesulfonyl)-L-arginyl]-2-piperidinecarboxylic acid monohydrate (hereinafter occasionally referred to as “argatroban”).

Examples of the pharmaceutically acceptable salt include, for example, base addition salts such sodium salts, potassium salts, calcium salts, ammonium salts, and amine salts, mineral acid salts such as hydrochlorides, hydrobromides, hydroiodides, sulfates, nitrates, and phosphates, organic acid salts such as acetates, maleates, fumarates, citrates, oxalates, succinates, tartrates, malates, mandates, methanesulfonates, p-toluenesulfonates, and 10-camphorsulfonates.

A route of administration of the auxiliary medicament for ophthalmic operation of the present invention is not particularly limited. An example includes intravenous administration by injection or drip infusion. As the auxiliary medicament for ophthalmic operation of the present invention, the compound of the aforementioned formula, per se, may be used as an active ingredient. Preferably, the medicament may be provided as formulations well known to one of ordinary skill in the art by adding a suitable pharmacologically and pharmaceutically acceptable additive to the compound of the aforementioned formula as an active ingredient. As the pharmacologically and pharmaceutically acceptable additives, for example, diluents, dissolving agents or dissolving aids, isotonic agents, pH modifiers, stabilizers and the like may be used.

For formulations suitable for injections or drip infusions, pharmaceutical additives may be added which include, for example, dissolving agents or dissolving aids which can constitute aqueous injections or injections dissolved before use such as water for injection, physiological saline, and propylene glycol, isotonic agents such as glucose, sodium chloride, D-mannitol, and glycerol, pH modifiers such as inorganic acid or organic acids, inorganic bases, or organic bases.

The auxiliary medicaments for ophthalmic operation of the present invention may be directly administered intraocularly or as intraocular irrigating solutions or eye drops.

The intraocular irrigating solution of the present invention can be prepared by dissolving the compound of the present invention in a sterilized purified water and the like. Pharmaceutically acceptable additives such as isotonic agents, buffering agents and the like may be added, if necessary, so that the solution becomes close to the aqueous humor composition. Specifically, glucose, sodium chloride, potassium chloride, calcium chloride, magnesium sulfate, sodium bicarbonate and the like may be added.

For use as eye drops, examples of the formulations include aqueous eye drops, non-aqueous eye drops, suspension-type eye drops, emulsion-type eye drops and the like. The eye drops may be prepared by using sterilized purified water, physiological salt solution and the like as aqueous mediums or vegetable oil such as cottonseed oil, soybean oil, sesame oil, and peanut oil and the like as non-aqueous mediums, and dissolving or suspending the compound of the present invention in the mediums. For the preparation, pharmaceutically acceptable additives such as isotonic agents, pH modifiers, thickeners, suspending aids, emulsifiers, and preservatives and the like may be added, if necessary. Specific examples of the isotonicities include sodium chloride, boric acid, sodium nitrate, potassium nitrate, D-mannitol, glucose and the like. Examples of the pH modifiers include boric acid, anhydrous sodium sulfate, hydrochloric acid, citric acid, sodium citrate, acetic acid, potassium acetate, sodium carbonate, sodium borate and the like. Examples of the thickeners include methylcellulose, hydroxypropyl methylcellulose, polyvinyl alcohol, sodium chondroitin sulfate, polyvinylpyrrolidone and the like. Examples of the suspending aids include polysorbate 80, polyoxyethylene hardening castor oil 60, polyoxy hardening castor oil and the like. Examples of the emulsifiers include yolk lecithin, polysorbate 80 and the like. Examples of the preservatives include benzalkonium chloride, benzethonium chloride, chlorobutanol, phenylethyl alcohol, paraoxybenzoic acid esters and the like.

An antithrombin agent or an antithrombotic agent (for injection) comprising which comprises (2R,4R)-4-methyl-1-[N²((RS)-3-methyl-1,2,3,4-tetrahydro-8-quinolinesulfonyl)-L-arginyl]-2-piperidinecarboxylic acid monohydrate as an active ingredient have already been used clinically (generic name “argatroban”; tradename “Novastan”, manufactured and distributed by Mitsubishi-Tokyo Pharmaceuticals, Inc.; or tradename “Slonnon”, bulk supplied by Mitsubishi-Tokyo Pharmaceuticals, Inc. and manufactured and distributed by Daiichi Pharmaceutical Co., Ltd.). The above marketed drugs, per se, may be used as the auxiliary medicament for ophthalmic operation of the present invention.

A dose of the auxiliary medicament for ophthalmic operation of the present invention is not limited, and the dose may suitably chosen depending on conditions such as route of administration, and the age, symptoms, body weight of a patient. For example, for intravenous administration, 0.1 to 50 mg/kg, preferably 0.4-3 mg/kg may be used per day for an adult as a weight of the active ingredient. For ophthalmic administration, an eye drop of about 1 mg/ml to 6 mg/ml can be used. For addition into an irrigating solution, concentration of the compound in the irrigating solution may be 0.1 μg/ml to 0.6 mg/ml.

The auxiliary medicament for ophthalmic operation of the present invention suppresses easy blood clotting and prevent formation of clotting mass without enhancing intraoperative bleeding. Accordingly, the medicament can be used as preventive medicament for easy clotting of intraoperative bleeding. The medicament can also be used as an auxiliary medicament for treatment of clotting mass which is generated intraoperatively during vitreous surgery for proliferating diabetic retinopathy and adhered firmly to the retina. Furthermore, treatment for intraoperative bleeding becomes easier, so that loads of a patient and an operator can be reduced and incidence of intraoperative iatrogenic retinal tear can be lowered.

### EXAMPLE

The present invention will be more specifically explained by referring to an example. However, as for as within the gist of the present invention, the present invention is not limited to the following example. As argatroban in the following example, a drug commercially available as the tradename “Slonnon” was used.

### Example 1

The patient was a youth suffering from diabetic retinopathy with increased intrinsic coagulation tendency. Fibrous vascular proliferating membrane and haemorrhagia praeretinalis resulting therefrom were observed in the left eye eyegrounds. Argatroban 10 mg as one ample was added to a drip infusion (specify ingredients, and a tradename and name of a manufacturing company if commercially available) and systemically administered to the patient from the beginning of the operation. The operation was completed for 2 hours. As for a rate for drip infusion during the operation, some bleedings were observed at treatment of fibrous vascular proliferating membrane, however, all of the bleedings did not give preretinal clotting mass and successfully removed by passive suction using a back flash needle. No abnormal bleeding tendency was not observed.

The above patient was a youth suffering from diabetic retinopathy with increased intrinsic coagulation tendency, and if treated under an ordinary operation, intraoperative bleedings would have expected to be easy clotting and caused operative difficulties. By administration of argatroban, treatment of intraoperative bleeding became easier, and no intraoperative iatrogenic tear was caused during treatment of fibrous vascular proliferating membrane.

Therefore, the aforementioned result shows that argatroban can be used as an auxiliary medicament for ophthalmic operation such as for intraoperative bleeding treatment during vitreous surgery for proliferation diabetic retinopathy and the like.

Industrial Applicability

The auxiliary medicament for ophthalmic operation of the present invention suppresses easy blood clotting and prevent formation of clotting mass without enhancing intraoperative bleeding. Accordingly, treatment for intraoperative bleeding becomes easier, so that loads of a patient and an operator can be reduced and incidence of intraoperative iatrogenic retinal tear is expected to be lowered.

The present application was filed by claiming priority on Japanese Patent Application No. 2000-18682.

What is claimed is:

1. An auxiliary method for an ophthalmic operation, in which (2R,4R)-4-methyl-1-[N²((RS)-3-methyl-1,2,3,4-tetrahydro-8-quinolinesulfonyl)-L-arginyl]-2-piperidinecarboxylic acid or a hydrate thereof, or a pharmaceutically acceptable salt thereof is used.

2. The auxiliary method for an ophthalmic operation according to claim 1, wherein the compound is (2R,4R)-4-methyl-1-[N²((RS)-3-methyl-1,2,3,4-tetrahydro-8-quinolinesulfonyl)-L-arginyl]-2-piperidinecarboxylic acid monohydrate.

3. The auxiliary method for an ophthalmic operation according to claim 1 or claim 2, wherein the compound is an auxiliary medicament for treatment of coagulation mass intraoperatively generated during an ophthalmic operation.

4. The auxiliary method for an ophthalmic operation according to any one of claims 1 to 3, wherein the ophthalmic operation is for proliferating diabetic retinopathy.

5. The auxiliary method for an ophthalmic operation according to any one of claims 1 to 4, wherein the ophthalmic operation is a vitreous surgery.

6. The auxiliary method for an ophthalmic operation according to any one of claims 1 to 5, wherein the compound is administered as an intraocular irrigating solution or an eye drop.

7. A method for preventing easy clotting of intraoperative bleeding by using (2R,4R)-4-methyl-1-[N²((RS)-3-methyl-1,2,3,4-tetrahydro-8-quinolinesulfonyl)-L-arginyl]-2-piperidinecarboxylic acid or a hydrate thereof, or a pharmaceutically acceptable salt thereof.

8. The method for preventing easy clotting of intraoperative bleeding according to claim 7, wherein the compound is (2R,4R)-4-methyl-1-[N²((RS)-3-methyl-1,2,3,4-tetrahydro-8-quinolinesulfonyl)-L-arginyl]-2-piperidinecarboxylic acid monohydrate.

9. The method for preventing easy clotting of intraoperative bleeding according to claim 7 or 8, which is used for treatment of coagulation mass intraoperatively generated during an ophthalmic operation.

10. The method for preventing easy clotting of intraoperative bleeding according to any one of claims 7 to 9, wherein the ophthalmic operation is for proliferating diabetic retinopathy.

11. The method for preventing easy clotting of intraoperative bleeding according to any one of claims 7 to 10, wherein the ophthalmic operation is a vitreous surgery.

12. The method for preventing easy clotting of intraoperative bleeding according to any one of claims 7 to 11, wherein the compound is administered as an intraocular irrigating solution or an eye drop.

13. A method for controlling bleeding during an ophthalmic operation by using (2R,4R)-4-methyl-1-[N²((RS)-3-methyl-1,2,3,4-tetrahydro-8-quinolinesulfonyl)-L-arginyl]-2-piperidinecarboxylic acid or a hydrate thereof, or a pharmaceutically acceptable salt thereof.

14. A method for reducing an incidence of intraoperative iatrogenic retinal tear by using (2R,4R)-4-methyl-1-[N²((RS)-3-methyl-1,2,3,4-tetrahydro-8-quinolinesulfonyl)-L-arginyl]-2-piperidinecarboxylic acid or a hydrate thereof, or a pharmaceutically acceptable salt thereof.
